(19) United States
(12) Patent Application Publication
(10) Pub. No.: US 2017/0000720 A1
(43) Pub. Date: Jan. 5, 2017

(54) COMPOSITION FOR EXTERNAL APPLICATION TO SKIN

(71) Applicant: ROHTO PHARMACEUTICAL CO., LTD., Osaka-shi (JP)

(72) Inventors: Sachie FUJITA, Osaka-shi (JP); Emi TAKEMOTO, Osaka-shi (JP)

(73) Assignee: ROHTO PHARMACEUTICAL CO., LTD., Osaka-shi (JP)

(21) Appl. No.: 15/107,399

(22) PCT Filed: Dec. 24, 2014
(86) PCT No.: PCT/JP2014/084030

§ 371 Date: Jun. 22, 2016

(30) Foreign Application Priority Data
Dec. 25, 2013 (JP) 2013-267325

(51) Int. Cl. A61K 8/73; A61K 8/81; A61K 8/86; A61Q 1/00

(52) U.S. Cl. CPC A61K 8/735; A61Q 1/00; A61K 8/8182; A61K 8/86; A61K 8/73; A61K 2800/884; A61K 2800/5922

(57) ABSTRACT

The problem addressed by the present invention is to provide a composition for external application to the skin containing a salt-resistant polymer and an acidic polysaccharide. The composition does not cause balling up of a cosmetic on the skin when a cosmetic is applied on the composition after the composition is applied on the skin. The composition for external application to the skin contains (A) a salt-resistant polymer, (B) an acidic polysaccharide, and (C) an amphipathic compound represented by the general formula (1): Z—[O-{(EO)ₐ(PO)_{b}}-(AO)_{c}—H]ₙ (1) wherein n, a, b, and c each represent a specific number; and Z, EO, PO, and AO each represent a specific group.

### TECHNICAL FIELD

The present invention relates to a composition for external application to the skin that contains a salt-resistant polymer, an acidic polysaccharide, and a specific amphipathic compound.

### BACKGROUND ART

Various polymers have thickening properties, and are capable of imparting an appropriate amount of viscosity to compositions, and thus are widely used in the field of compositions for external application to the skin, such as cosmetics, in order to enhance adhesion to the skin and to maintain favorable skin quality. Furthermore, an acidic polysaccharide is an ingredient having high water retention ability and widely used in various compositions for external application to the skin.

The above-described polymers might lose viscosity with time, for example, by being affected by another ingredient, such as salt. Among them, however, salt-resistant polymers are useful thickeners that can maintain stability (viscosity) of preparations even in the presence of an electrolyte.

Patent Literature 1 discloses a cosmetic composition in the form of an oil-in-water type emulsion, which contains an oil phase and a water phase. The composition is characterized by containing at least a thickener, at least an oil in an amount of at least 3% by weight relative to the total weight of the composition, and at least an oxyalkylenated derivative represented by the following formula (I):

Z—{O(AO)ₗ(EO)ₘ—(BO)ₙH}ₐ (I)

wherein Z denotes a group obtained by removing one or more hydroxy groups from a compound containing 3 to 9 hydroxyl groups; AO denotes an oxyalkylenated group containing 3 to 4 carbon atoms; EO denotes an oxyethylene group; BO denotes an oxyalkylene group containing 4 carbon atoms; a is in the range of 3 to 9; l, m, and n respectively denote an average molar number of AO, EO, and BO units, and are respectively 1≦1≦50, 1≦m≦50, and 0.5≦n≦5; and the weight ratio of AO to EO (AO/EO) is in the range of 1/5 to 5/1.

As a thickener in the invention according to Patent Literature 1, salt-resistant polymers, such as a polymer and a copolymer of 2-acrylamido-2-methylpropanesulfonic acid, are mentioned, and as an oxyalkylenated derivative of the formula (I), Wilbride S-753 is mentioned. In the Literature, xanthane gum is also mentioned as a thickener, and sodium hyaluronate is mentioned as an active ingredient usable in the invention of the Literature.

Patent Literature 1, however, neither discloses nor suggests the possibility of some problem occurring when a salt-resistant polymer is used with xanthane gum, or sodium hyaluronate.

### PRIOR ART LITERATURES

### Patent Literatures

[Patent Literature 1] JP 2011-32273 A

### SUMMARY OF THE INVENTION

### Problem to be Solved by the Invention

In a makeup application, cosmetics having various functions are generally applied in layers on the skin. If a cosmetic is applied on a composition for external application to the skin that contains a salt-resistant polymer and an acidic polysaccharide, like the one described above, after the composition is applied to the skin, the cosmetics are balled up on the skin.

Such a problem causes discomfort to the user of the composition and cosmetics, failing to achieve the purposes of cosmetics, including making the body of the wearer clean, beautiful, and more attractive, or changing the appearance of the wearer.

Thus, an object of the present invention is to provide a composition for external application to the skin that contains a salt-resistant polymer and an acidic polysaccharide, and that does not cause balling up of a cosmetic on the skin, when the cosmetic is applied on the composition for external application to the skin after the composition is applied to the skin.

### Means for Solving the Problem

As a result of intensive study to solve the problem, the present inventors found that the problem can be solved by mixing a specific amphipathic compound into a composition for external application to the skin that contains a salt-resistant polymer and an acidic polysaccharide.

That is, the gist of the present invention is as follows.

<1> A composition for external application to the skin comprising: (A) a salt-resistant polymer; (B) an acidic polysaccharide; and (C) an amphipathic compound represented by the following general formula (1):

Z—[O-{(EO)ₐ(PO)_{b}}-(AO)_{c}—H]ₙ (1)

wherein:
n is an integer of 1 to 10;
Z is a hydrogen atom, or a group obtained by removing n number of hydroxy group(s) from a hydroxy compound of a carbon number of 1 to 30;
EO is an ethylene oxide group;
PO is a propylene oxide group;
AO is an alkylene oxide group of a carbon number of 2 to 5;
a, b, and c each denote an average addition molar number of the respective alkylene oxide groups, and are independently 0 to 200; with the proviso that not all of a, b, and c are 0 at the same time. Furthermore, when n is 2 or more, the respective pluralities of a's, b's, c's, and AOs may be the same as or different from one another, and when n is 3, the carbon number of AO is 2, 3, or 5. When Z is a hydrogen atom, n is 1.
Note that in { }, the respective alkylene oxide groups may be added randomly or in blocks.

<2> The composition for external application to the skin according to <1>, wherein the amphipathic compound is a compound represented by the following general formula (2):

Z—[O-{(EO)ₐ(PO)_{b}}—H]ₙ (2)

wherein:
n is an integer of 1 to 10;
Z is a hydrogen atom, or a group obtained by removing n number of hydroxy group(s) from a hydroxy compound of a carbon number of 1 to 30;
EO is an ethylene oxide group;
PO is a propylene oxide group;
a and b each denote an average addition molar number of the respective alkylene oxide groups, and are independently 0 to 200; with the proviso that not all of a and b are 0 at the same time. Furthermore, when n is 2 or more, the respective pluralities of a's and b's may be the same as or different from one another. When Z is a hydrogen atom, n is 1.
Note that in { }, the respective alkylene oxide groups may be added randomly or in blocks.

<3> The composition for external application to the skin according to <1> or <2>, wherein Z in the above-described general formula (1) or (2) is a hydrogen atom, or, a group obtained by removing n number of hydroxy group(s) from an alkyl monoalcohol of a carbon number of 4 to 24, glycerin, trimethylolpropane, erythritol, pentaerythritol, alkylglycoside, diglycerin, xylitol, dipentaerythritol, sorbitol, inositol, sucrose, trehalose, or maltitol.

<4> The composition for external application to the skin according to any of <1> to <3>, wherein Z in the above-described general formula (1) or (2) is a hydrogen atom, or a group obtained by removing a hydroxy group from an alkyl monoalcohol of a carbon number of 4 to 24, a group obtained by removing 1 to 6 hydroxy groups from sorbitol, or a group obtained by removing 1 to 4 hydroxy groups from diglycerin.

<5> The composition for external application to the skin according to any of <1> to <4>, wherein the acidic polysaccharide (B) is an acidic mucopolysaccharide, xanthane gum, or gellan gum.

<6> The composition for external application to the skin according to any of <1> to <5>, wherein the salt-resistant polymer (A) is a polymer having, on a side chain thereof, dimethyl taurine or a salt thereof.

<7> The composition for external application to the skin according to <6>, wherein the salt-resistant polymer (A) is at least one selected from the group consisting of (hydroxyethyl acrylate/Na acryloyldimethyltaurate) copolymer, (ammonium acryloyldimethyltaurate/vinylpyrrolidone) copolymer, (ammonium acryloyldimethyltaurate beheneth-25 methacrylate) cross-copolymer, (ammonium acryloyldimethyltaurate/steareth-25 methacrylate) crosspolymer, and (acrylic acid/acryloyldimethyltaurate/dimethylacrylamide) crosspolymer.

<8> A method for applying makeup, comprising applying to the skin the composition for external application to the skin according to any of <1> to <7>, and subsequently applying a makeup cosmetic on the skin.

### Effect of the Invention

The present invention provides a composition for external application to the skin that contains a salt-resistant polymer and an acidic polysaccharide, and that is still capable of avoiding balling up of cosmetics on the skin when cosmetics are applied in layers on the composition for external application to the skin after the composition is applied to the skin.

### BRIEF DESCRIPTION OF THE DRAWINGS

FIG. 1 shows photographs of representative conditions corresponding to the scores evaluated in five scales of test examples in Examples.

### DESCRIPTION OF THE INVENTION

Hereinafter, the present invention will be described in detail.

[Composition for External Application to the Skin]

The composition for external application to the skin of the present invention contains, as described above, (A) a salt-resistant polymer; (B) an acidic polysaccharide; and (C) an amphipathic compound represented by the above-described general formula (1). Hereinafter, these components and various other components or the like that may be added to the composition for external application to the skin of the present invention will be described.

<(A) Salt-Resistant Polymer>

The salt-resistant polymer (A) is an ingredient used for imparting appropriate viscosity to the composition for external application to the skin of the present invention. Furthermore, the viscosity of the salt-resistant polymer (A) is less likely to be affected by an electrolyte or the like, so that various ingredients can be added as desired to adjust various properties, such as pH, suitable for the application to the skin while maintaining the viscosity.

In the present invention, various publicly known polymers that can maintain the viscosity of preparations even in the presence of an electrolyte can be used as a salt-resistant polymer (A).

Although such a salt-resistant polymer (A) is not particularly limited, a polymer having, on a side chain thereof, dimethyl taurine or a salt thereof is preferable with the object of maintaining appropriate viscosity and imparting fresh feeling.

The dimethyl taurine or a salt thereof that forms at least a portion of a side chain is a group having a structure represented by the following formula (3):

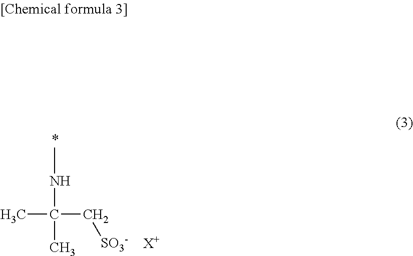

In the formula, X⁺ is a cation. Furthermore, * is a bonding hand that bonds directly or indirectly to the main chain of the above-described polymer.

Examples of the cation represented by the X⁺ include a proton, an alkali metal cation, and an ammonium ion. Of these, a proton, a sodium ion, and an ammonium ion are preferable. It should be noted that when the cation represented by X⁺ is a proton, then the salt-resistant polymer (A) has, on a side chain thereof, dimethyl taurine.

Examples of the polymer having, on a side chain thereof, dimethyl taurine or a salt thereof as described above include a homopolymer composed of monomers that form, on a side chain thereof, dimethyl taurine or a salt thereof, a copolymer composed of two or more types of such monomers, or a copolymer composed of such monomers and other monomers that do not form, on a side chain thereof, dimethyl taurine or a salt thereof.

Specific examples of the monomers that form, on a side chain thereof, dimethyl taurine or a salt thereof include acryloyldimethyl taurine, sodium acryloyldimethyltaurate, and ammonium acryloyldimethyltaurate.

Furthermore, examples of the other monomers include vinyl-based monomers having a vinyl group. The vinyl-based monomers may also be (meth)acryl-based monofunctional monomers. As a suitable example of vinyl-based monomers, vinyl monomers having a functional group (pyrrolidinyl group) represented by the following formula (4) can be mentioned.

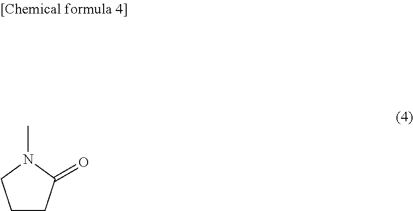

More specifically, N-vinyl-2-pyrrolidone may be mentioned as a monomer having a functional group represented by the above-described formula (4).

Another suitable example of the vinyl-based monomer is a (meth)acrylic acid-based monofunctional monomer, and among them, a methacrylic acid-based monofunctional monomer may be mentioned. In particular, a monomer represented by the following general formula (5) is preferable.

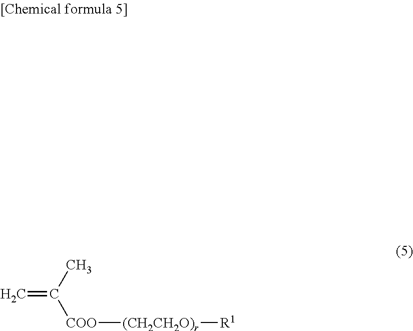

wherein R¹ denotes an alkyl group of a carbon number of 1 to 30, and r denotes an integer of 1 to 50.

In the formula (5), although R¹ may be any alkyl group of a carbon number of 1 to 30 without particular limitation, R¹ is preferably a methyl group, an ethyl group, a stearyl group, or a behenyl group, more preferably a stearyl group or a behenyl group, and most preferably a behenyl group. Furthermore, although r may be any integer of 1 to 50 without particular limitation, r is preferably an integer of 1 to 40, more preferably an integer of 1 to 30, and most preferably an integer of 10 to 30.

Such other monomers may be copolymerized with a salt-resistant polymer (A) in a proportion that does not impair the salt-resistance of the salt-resistant polymer (A) so as to impart other desired properties.

Specific examples of the above-described salt-resistant polymer (A) include (hydroxyethyl acrylate/Na acryloyldimethyltaurate) copolymer, (ammonium acryloyldimethyltaurate/vinylpyrrolidone) copolymer, (ammonium acryloyldimethyltaurate/beheneth-25 methacrylate) cross-copolymer, (ammonium acryloyldimethyltaurate/steareth-25 methacrylate) crosspolymer, and (acrylic acid/acryloyldimethyltaurine/dimethylacrylamide) crosspolymer.

The above-described salt-resistant polymer (A) may be obtained by synthesis or may be acquired as a commercial product. As a commercial product, for example, Aristoflex AVC (trade name), Aristoflex HMB (trade name), or Aristoflex HMS (trade name) [all manufactured by Clariant (Japan) K.K.], SEPINOV EMT 10 (trade name), SIMULGEL NS (trade name), SIMULGEL FL (trade name), SEPIPLUS S (trade name), or SEPINOV P88 (trade name) [all manufactured by SEPPIC Inc.] can be suitably used.

In the present invention, a single type of a salt-resistant polymer (A) may be used alone, or two or more types of salt-resistant polymers (A) may be used in combination. The content of the salt-resistant polymer (A) in the entire composition for external application to the skin of the present invention (in 100% by weight) is 0.01 to 5% by weight, preferably 0.05 to 4% by weight, and more preferably 0.1 to 3% by weight with the object of maintaining appropriate viscosity etc.

<(B) Acidic Polysaccharide>

The acidic polysaccharide (B) is an ingredient used to impart superior water retention ability to the composition for external application to the skin of the present invention. In the present invention, various publicly known polysaccharides having an acidic group may be used without particular limitation as an acidic polysaccharide (B). Furthermore, as the acidic group, a carboxylic acid group and a sulfonic acid group may be mentioned.

As the acidic polysaccharide (B), acidic mucopolysaccharides, xanthane gum, and gellan gum are preferable with the object of imparting water retention ability, smoothness in application, and appropriate slight stickiness. Examples of the acidic mucopolysaccharides include hyaluronic acid, chondroitin, chitosan, jaluron acid, heparan, keratin, and alginic acid, as well as derivatives or salts thereof.

Examples of the above-described derivatives include acetylated products. Furthermore, as the above-described salts, any pharmaceutically or biologically acceptable salt may be used, and, for instance, alkali metal salts, such as sodium and potassium; alkaline-earth metal salts, such as magnesium and calcium; zinc salts; ammonium salts; and alkanolamine salts, such as monoethanolamine, may be mentioned. Of these, alkali metal salts are preferable, and a sodium salt is more preferable.

Specifically preferred examples of the salts of acidic mucopolysaccharides include sodium hyaluronate, potassium hyaluronate, calcium hyaluronate, magnesium hyaluronate, zinc hyaluronate, ammonium hyaluronate, monoethanolamine hyaluronate, acetylated sodium hyaluronate, acetylated potassium hyaluronate, acetylated calcium hyaluronate, acetylated magnesium hyaluronate, acetylated zinc hyaluronate, acetylated ammonium hyaluronate, acetylated ethanolamine hyaluronate, chondroitin sodium sulfate, chondroitin potassium sulfate, dermatan sodium sulfate, dermatan potassium sulfate, chitosan ascorbate, chitosan glycolate, chitosan lactate, chitosan hydroxypropyltrimonium chloride, heparansodium sulfate, and heparan potassium sulfate.

Of the acidic mucopolysaccharides, hyaluronic acid, sodium hyaluronate, chondroitin sulfate, chondroitin sodium sulfate, acetylated sodium hyaluronate, acetylated potassium hyaluronate, acetylated calcium hyaluronate, acetylated magnesium hyaluronate, acetylated zinc hyaluronate are preferable; hyaluronic acid, sodium hyaluronate, chondroitin sulfate, chondroitin sodium sulfate, acetylated sodium hyaluronate, acetylated potassium hyaluronate, and acetylated zinc hyaluronate are more preferable; and hyaluronic acid and sodium hyaluronate are still more preferable.

A single type of an acidic mucopolysaccharide may be used alone, or two or more types of acidic mucopolysaccharides may be used in combination. An acid mucopolysaccharide of any derivation may be used without particular limitation, and any one usable in the field of pharmaceutical products, quasi-drugs, or cosmetics may be used.

The average molecular weight of the acidic mucopolysaccharides is not particularly limited, and, for instance, about 1000 to 4 million, preferably about 1000 to 3 million, more preferably about 5000 to 3 million, and particularly preferably about 5000 to 2 million.

Among these, as an acidic polysaccharide (B), hyaluronic acid or a derivative thereof, or salts thereof is particularly preferable with the object of imparting water retention ability and smoothness in application and appropriate slight stickiness.

In the present invention, a single type of an acidic polysaccharide (B) may be used alone, or two or more types of acidic polysaccharides (B) may be used in combination. The content of the acidic polysaccharide (B) in the entire composition for external application to the skin of the present invention (in 100% by weight) is 0.001 to 1% by weight, preferably 0.01 to 0.8% by weight, and more preferably 0.05 to 0.5% by weight with the object of water retention ability.

Furthermore, the mixing ratio of the acidic polysaccharide (B) to the salt-resistant polymer (A) is as follows: the acidic polysaccharide (B) is usually 0.001 to 100 parts by weight, preferably 0.005 to 50 parts by weight, and more preferably 0.01 to 10 parts by weight relative to one part by weight of the salt-resistant polymer (A).

<(C) Amphipathic Compound>

The above-described ingredient (A) and ingredient (B) are ingredients widely used in compositions for external application to the skin. It became clear this time, however, that when a composition containing these ingredients is applied to the skin, for instance, as a base, and then cosmetics are subsequently applied thereon, the cosmetics may be balled up on the skin.

In the present invention, mixing a specific amphipathic compound (C) in addition to the ingredient (A) and the ingredient (B) prevents occurrence of balling up of a cosmetic on the skin. The amphipathic compound (C) is represented by the following general formula (1):

Z—[O-{(EO)ₐ(PO)_{b}}-(AO)_{c}—H]ₙ (1)

In the general formula (1), n is an integer of 1 to 10; Z is a hydrogen atom, or a group obtained by removing n number of hydroxy group(s) from a hydroxy compound of a carbon number of 1 to 30; EO is an ethylene oxide group; PO is a propylene oxide group; AO is an alkylene oxide group of a carbon number of 2 to 5; a, b, and c each denote an average addition molar number of the respective alkylene oxide groups, and are independently 0 to 200; with the proviso that not all of a, b, and c are 0 at the same time. Furthermore, when n is 2 or more, the respective pluralities of a's, b's, c's, and AOs may be the same as or different from one another, and when n is 3, the carbon number of AO is 2, 3, or 5. When Z is a hydrogen atom, n is 1. Furthermore, the respective alkylene oxide groups in { } in formula (1) may be added randomly or in blocks. Furthermore, when a, b, and c each are other than 0, a, b, and c are preferably independently 1 to 200, and more preferably 2 to 150.

In the amphipathic compound (C), OH at the right end of the general formula (1) and OH including Z (when Z is a hydrogen atom) form a hydrophilic portion, whereas Z (when Z is obtained as a group by removing n number of hydroxy group(s) from a hydroxy compound of a carbon number of 1 to 30), PO, and AO form a hydrophobic portion. Furthermore, EO, which is relatively hydrophilic compared to PO and EO, may function as a hydrophilic portion.

The amphipathic compound is preferably a compound represented by the following general formula (2) with the object of maintaining the amphipathic property while preventing balling up of a cosmetic on the skin due to the amphipathic property.

Z—[O-{(EO)ₐ(PO)_{b}}—H]ₙ (2)

In the formula, n, Z, EO, PO, a, and b are as defined with regard to the general formula (1), and the respective alkylene oxide groups in { } of the general formula (2) may be added randomly or in blocks. Furthermore, not all of a and b are 0 at the same time, and when n is 2 or more, the respective pluralities of a's and b's may be the same as or different from one another, and when Z is a hydrogen atom, n is 1. Furthermore, when a and b each are other than 0, a and b are preferably independently 1 to 200, and more preferably 2 to 150.

The amphipathic compound (C) used in the present invention is a polymer of a specific alkylene glycol (when Z is a hydrogen atom), or a compound obtained by adding a specific alkylene oxide group (corresponding to a portion of general formulas (1) and (2) excluding Z) to a specific hydroxy compound (corresponding to Z) (when Z is a group obtained by removing n number of hydroxy group(s) from a hydroxy compound having 1 to 30 carbons).

In the general formulas (1) and (2), AO is preferably an alkylene oxide group of a carbon number of 2 to 4, and a, b, and c are preferably each independently 1 to 150 with the object of preventing balling up of a cosmetic on the skin or the like.

Furthermore, for the same object, Z is preferably a hydrogen atom, or a group obtained by removing n number of hydroxy group(s) from an alkyl monoalcohol of a carbon number of 4 to 24, glycerin, trimethylolpropane, erythritol, pentaerythritol, alkylglycoside, diglycerin, xylitol, dipentaerythritol, sorbitol, inositol, sucrose, trehalose, or maltitol. That is, Z is preferably a group derived from these alcohol compounds, and is more preferably a hydrogen atom, or a group derived from an alkyl monoalcohol of a carbon number of 4 to 24, sorbitol (having 6 hydroxy groups to which an alkylene oxide group can be added) or diglycerin (having 4 hydroxy groups to which an alkylene oxide group can be added).

Such an amphipathic compound (C) can be synthesized by a publicly known method, and is also commercially available in the trade names such as UNILUBE 50MB-26 (PPG-17 buteth-17) (manufactured by NOF Corporation), UNIOL HS-1600D (PPG-25 sorbitol) (manufactured by NOF Corporation), Pronon No. 208 (PEG/PPG-150/35 copolymer) (manufactured by NOF Corporation), SY-DP14T (PPG-14 polyglyceryl-2 ether) (manufactured by Sakamoto Yakuhin Kogyo Co., Ltd.), SY-DP9 (PPG-9 diglyceryl) (manufactured by Sakamoto Yakuhin Kogyo Co., Ltd.), UNIOL D-2000 (PPG-34) (manufactured by NOF Corporation), EMULGEN PP-290 (PEG/PPG-160/30 copolymer) (manufactured by Kao Corporation), New Pole GP-1000 (PPG-16 glyceryl ether) (manufactured by Sanyo Chemical Industries, Ltd.), New Pole SP-750 (PPG-10 sorbitol) (manufactured by Sanyo Chemical Industries, Ltd.), New Pole PE-68 (PEG-160/30 copolymer) (manufactured by Sanyo Chemical Industries, Ltd.), and New Pole PE-78 (PEG-150/35 copolymer) (manufactured by Sanyo Chemical Industries, Ltd.).

In the present invention, a single type of an amphipathic compound (C) may be used alone, or any two or more types of amphipathic compounds (C) may be used in combination. The content of the amphipathic compound (C) in the entire composition for external application to the skin of the present invention (in 100% by weight) is 0.01 to 15% by weight, preferably 0.05 to 10% by weight, and more preferably 0.1 to 5% by weight with the object of preventing balling up of a cosmetic on the skin or the like.

Furthermore, the mixing ratio of the salt-resistant polymer (C) to the amphipathic compound (A) is as follows: the amphipathic compound (C) is usually 1 to 1000 parts by weight, preferably 1 to 500 parts by weight, and more preferably 1 to 50 parts by weight relative to one part by weight of the salt-resistant polymer (A).

The mixing ratio of the acidic polysaccharide (C) to the amphipathic compound (B) is as follows: the amphipathic compound (C) is usually 1 to 5000 parts by weight, preferably 1 to 1000 parts by weight, and more preferably 1 to 500 parts by weight relative to one part by weight of the acidic polysaccharide (B).

<Other Ingredients>

To impart other useful effects, in addition to the above-described ingredients, a single one of, or two or more in combination of various ingredients such as a UV scattering ingredient, a UV absorbing ingredient, an ingredient having a DNA damage preventing and/or repairing effect, a whitening ingredient, an ingredient having an anti-inflammatory effect, an antibacterial ingredient, a cell-activating ingredient, an astringent ingredient, an anti-oxidative ingredient, an anti-aging ingredient, a moisturizing ingredient, a keratin-softening ingredient, vitamins, a blood circulation-promoting ingredient, and a sebum-adsorbing ingredient may be mixed to the composition for external application to the skin of the present invention. Any of these ingredients that can be used in the fields of pharmaceutical products, quasi-drugs, and cosmetics may be used without particular limitation, and may be appropriately selected for use. Furthermore, any ingredient that corresponds to a plurality of ingredients shown below may be added to impart any of the effects of the ingredient.

Examples of the above-described UV scattering ingredient include inorganic compounds, such as zinc oxide, titanium oxide, iron oxide, cerium oxide, zirconium oxide, titanium silicate, zinc silicate, silicic anhydride, cerium silicate, and hydrated silicate; the inorganic compounds coated with an inorganic powder such as hydrated silicate, aluminium hydroxide, mica, and talc; and composites of the compounds with a resin powder such as polyamide, polyethylene, polyester, polystyrene, and nylon, and those further treated with silicon oil, fatty acid aluminum salt, etc. Of these, the inorganic compounds such as zinc oxide, titanium oxide, and iron oxide, and the inorganic compounds coated with an inorganic powder, such as aluminium hydroxide, hydrated silicate, mica, and talc, and silicon oil are preferable. In the case where a UV scattering ingredient is mixed, the content used may be appropriately selected in consideration of the feeling of use and the effect when applied to the skin. The content is, for instance, about 0.001 to 35% by weight, and preferably about 0.1 to 25% by weight relative to the entire composition for external application to the skin of the present invention.

Examples of the above-described UV absorbing ingredient include 2-ethylhexyl-p-methoxycinnamate, 2-[4-(diethylamino)-2-hydroxybenzoyl] benzoic acid hexyl ester, 2,4,6-tris[4-(2-ethylhexyloxycarbonyl)anilino]-1,3,5-triazine, 2-ethylhexyl-dimethoxy benzylidene oxo imidazolidine-propionate, and 2,4-bis-[{4-(2-ethylhexyloxy)-2-hydroxy}phenyl]-6-(4-methoxyphenyl)-1,3,5-triazine. In the case where a UV absorbing ingredient is mixed, the content used may be appropriately selected in consideration of the feeling of use and the effect when applied to the skin. The content is, for instance, about 0.01 to 20% by weight, and preferably about 0.1 to 15% by weight relative to the entire composition for external application to the skin of the present invention.

Examples of the above-described ingredient having a DNA damage preventing and/or repairing effect include ingredients derived from animals (e.g., Artemia); ingredients derived from plants (e.g., Cat's Claw); and nucleic acid ingredients, such as DNA, salts of DNA, RNA, and salts of RNA. The content of the ingredient having a DNA damage preventing and/or repairing effect may be appropriately selected in consideration of the feeling of use and the effect when applied to the skin. The content is, for instance, about 0.001 to 3% by weight, and preferably about 0.01 to 1% by weight relative to the entire composition for external application to the skin of the present invention. When an animal ingredient or a plant ingredient is used, the content in extract is about 0.00001 to 0.1% by weight, and preferably about 0.0001 to 0.01% by weight relative to the entire composition for external application to the skin of the present invention.

Examples of the whitening ingredient include placenta; arbutin; kojic acid; ellagic acid; phytic acid tranexamic acid; rucinol; kamomilla ET; and vitamins, such as vitamin A or derivatives thereof, and pantothenic acid or derivatives thereof. Furthermore, a plant ingredient having a whitening effect may be used as a whitening ingredient. Examples of such a plant ingredient include ingredients derived from iris, almond, aloe, ginkgo, oolong tea, rose fruit, scutellaria root, Coptis Rhizome, St. John's wort (Hypericum erectum Thunb), dead nettle, seaweed, pueraria root, gardenia, Sophorae Radix, chlorella, gallnut, wheat, rice, rice germ, orizanol, rice bran, Asiasari Radix, zanthoxylum fruit, perilla, peony root, Cnidium Rhizome, mulberry bark, soybeans, fermented soybeans, tea, Japanese angelica, Calendula officinalis, garlic, hamamelis, safflower, moutan bark, Angelica acutiloba Kitagawa, amethyst, gambir, Japanese andromeda, Japanese andromeda, bracken, Podocarpus macrophyllus, Celtis sinensis, persimmon (Diospyros kaki), catalpa, black soybeans, gentian, figwort (Scrophularia ningpoensis), sarsaparilla, French beans, cimicifuga rhizome, Paris polyphylla Smith, sage, Peucedani Radix, Japanese radish, azalea, Lespedeza homoloba, tocin, Picrasma quassioides, parsley, hollies, hops, Lespedeza cyrtobotrya, cloves, and licorice. Preferred are ingredients derived from iris, aloe, ginkgo, oolong tea, rose fruit, scutellaria root, Coptis Rhizome, St. John's wort (Hypericum erectum Thunb), dead nettle, seaweed, pueraria root, gardenia, Sophorae Radix, gallnut, wheat, rice, rice bran, Asiasari Radix, zanthoxylum fruit, perilla, peony, Cnidium Rhizome, mulberry bark, tea, Japanese angelica, Calendula officinalis, hamamelis, safflower, Moutan bark, coix seeds, Angelica acutiloba Kitagawa, gambir, Celtis sinensis, persimmon (Diospyros kaki), catalpa, black soybeans, gentian, sarsaparilla, French beans, Paris polyphylla Smith, sage, Peucedani Radix, Japanese radish, azalea, Lespedeza homoloba, tocin, Picrasma quassioides, parsley, hollies, hops, cloves, licorice, and Japanese angelica. More preferred are ingredients derived from iris, aloe, ginkgo, rose fruit, scutellaria root, Coptis Rhizome, St. John's wort (Hypericum erectum Thunb), gardenia, Sophorae Radix, rice, rice bran, Asiasari Radix, peony, Cnidium Rhizome, mulberry bark, tea, Japanese angelica, Calendula officinalis, hamamelis, safflower, moutan bark, amethyst, Gambir, Celtis sinensis, persimmon (Diospyros kaki), sage, Japanese radish, azalea, parsley, hops, licorice, and coix seeds. When such a plant ingredient is used in the composition for external application to the skin of the present invention, the plant ingredient may be used in any form without particular limitation, and is usually in the form of a plant essence (plant extract) or an essential oil. Furthermore, the names given in the parentheses ( ) in the above plant ingredients are the scientific names, other names, or the names of crude drugs of the plants. In the case where one of the above-described whitening ingredients is mixed into the composition for external application to the skin of the present invention, the amount to be used can be appropriately selected in consideration of the feeling of use and the effect when applied to the skin, and is, for instance, about 0.0003 to 10% by weight, and preferably about 0.01 to 5% by weight relative to the entire composition for external application to the skin of the present invention. When a plant essence is used, the amount to be used in extract, such as essence, is about 0.00001 to 20% by weight, preferably about 0.0001 to 15% by weight, and more preferably 0.001 to 10% by weight relative to the entire composition for external application to the skin of the present invention.

Examples of the above-described anti-inflammatory ingredient include allantoin, calamine, tranexamic acid, glycyrrhizin acid or a derivative thereof, or salts thereof, glycyrrhetinic acid or a derivative thereof, or salts thereof, zinc oxide, guaiazulene, tocopherol acetate, pyridoxine hydrochloride, menthol, camphor, turpentine, indometacin, salicylic acid, or derivatives thereof. Preferred is glycyrrhizin acid or a derivative thereof, or a salt thereof (e.g., dipotassium glycyrrhizinate), glycyrrhetinic acid or a derivative thereof, or a salt thereof, or zinc oxide. In the case where an anti-inflammatory ingredient is mixed, the amount to be used can be appropriately selected in consideration of the feeling of use and the effect when applied to the skin, and is, for instance, about 0.0003 to 10% by weight, and preferably about 0.01 to 5% by weight relative to the entire composition for external application to the skin of the present invention.

Examples of the above-described antibacterial ingredient include chlorhexidine, salicylic acid, benzalkonium chloride, acrinol, ethanol, benzethonium chloride, cresol, gluconic acid and their derivatives, povidone iodine, potassium iodide, iodine, isopropyl methyl phenol, triclocarban, triclosan, Photosensitizing dye No. 101, Photosensitizing dye No. 201, paraben, phenoxyethanol, and hydrochloric alkyl diamino glycine. In the case where an antibacterial ingredient is mixed, the amount to be used can be appropriately selected in consideration of the feeling of use and the effect when applied to the skin, and is, for instance, about 0.0003 to 10% by weight, and more preferably about 0.01 to 5% by weight relative to the entire composition for external application to the skin of the present invention.

Examples of the above-described cell-activating ingredient include amino-acids, such as γ-amino butyric acid and ε-amino capronic acid; vitamins, such as retinol, thiamine, riboflavin, pyridoxine hydrochloride, and pantothenic acid; and α-hydroxy acids, such as glycolic acid and lactic acid; tannin, flavonoid, saponin, allantoin, and Photosensitizing dye No. 301. In the case where a cell-activating ingredient is mixed, the amount to be used can be appropriately selected in consideration of the feeling of use and the effect when applied to the skin, and is, for instance, about 0.0003 to 10% by weight, and preferably about 0.01 to 5% by weight relative to the entire composition for external application to the skin of the present invention.

Examples of the above-described astringent ingredient include metal salts, such as alum, chlorohydroxy aluminum, aluminium chloride, allantoin aluminium salt, zinc sulfate, and aluminum potassium sulfate; and organic acids, such as tannic acid, citric acid, lactic acid, and succinic acid. In the case where an astringent ingredient is mixed, the amount to be used can be appropriately selected in consideration of the feeling of use and the effect when applied to the skin, and is, for instance, about 0.0003 to 10% by weight, and preferably about 0.01 to 5% by weight relative to the entire composition for external application to the skin of the present invention.

Examples of the above-described antioxidative ingredient include butylated hydroxyanisole, dibutylhydroxytoluene, sodium hydrogen sulfite, sodium metabisulfite, erythorbic acid and a salt thereof, ascorbic acid and a salt thereof, flavonoid, glutathione, glutathione peroxidase, glutathione-S-transferase, catalase, super oxide dismutase, thioredoxin, taurine, thiotaurine, and hypotaurine. In the case where an antioxidative ingredient is mixed, the amount to be used can be appropriately selected in consideration of the feeling of use and the effect when applied to the skin, and is, for instance, about 0.00001 to 10% by weight, preferably about 0.0001 to 5% by weight, and more preferably 0.001 to 5% by weight relative to the entire composition for external application to the skin of the present invention.

Examples of the above-described anti-aging ingredient include retinoid (retinol, retinoic acid, retinal, etc.), pangamic acid, ursolic acid, turmeric essence, sphingosine derivatives, silicon, silicic acid, N-methyl-L-serine, and mevalonic lactone. In the case where an anti-aging ingredient is mixed, the amount to be used can be appropriately selected in consideration of the feeling of use and the effect when applied to the skin, and is, for instance, about 0.0003 to 10% by weight, and preferably about 0.01 to 5% by weight relative to the entire composition for external application to the skin of the present invention.

Examples of the above-described moisturizing ingredient include amino acids, such as alanine, serine, leucine, isoleucine, threonine, glycine, proline, hydroxyproline, glucosamine, and theanine, and derivatives thereof; polyhydric alcohols, such as glycerin; sugar alcohols, such as sorbitol; phospholipids, such as lecithin and hydrogenated lecitin; propylene glycol; and ingredients derived from NMF, such as lactate, sodium pyrrolidone carboxylate, and urea. In the case where a moisturizing ingredient is mixed, the amount to be used can be appropriately selected in consideration of the feeling of use and the effect when applied to the skin, and is, for instance, about 0.1 to 10% by weight, and preferably about 0.5 to 5% by weight relative to the entire composition for external application to the skin of the present invention.

Examples of the above-described keratin-softening ingredient include lanolin, urea, phytic acid, lactic acid, lactate, glycolic acid, salicylic acid, malic acid, citric acid, and gluconic acid. In the case where a keratin-softening ingredient is mixed, the amount to be used can be appropriately selected in consideration of the feeling of use and the effect when applied to the skin, and is, for instance, 0.0001 to 50% by weight, preferably about 0.001 to 50% by weight, and more preferably about 0.05 to 25% by weight relative to the entire composition for external application to the skin of the present invention.

Examples of the above-described vitamins include a group of vitamin A including retinol derivatives, such as retinol, retinol acetate, and retinol palmitate, retinal, retinoic acid, methyl retinoate, ethyl retinoate, retinol retinoate, d-δ-tocopheryl retinoate, α-tocopheryl retinoate, and β-tocopheryl retinoate; a group of provitamin A such as β-carotene, α-carotene, γ-carotene, δ-carotene, lycopene, zeaxanthin, cryptoxanthin, and echinenone; a group of vitamin E such as δ-tocopherol, α-tocopherol, β-tocopherol, dl-α-tocopherol succinate, dl-α-tocopherol calcium succinate, δ-tocopherol, and tocopherol nicotinate; a group of vitamin B2 such as riboflavin, flavin mononucleotide, flavin adenine dinucleotide, riboflavin butyric acid ester, riboflavin tetra-butyric acid ester, riboflavin 5′-phosphoric acid ester sodium, and riboflavin-tetra nicotinic acid ester; nicotinic acids, such as methyl nicotinate, nicotinic acid, and nicotinic acid amide; a group of vitamin C such as ascorbyl stearate, L-ascorbyl dipalmitate, ascorbyl tetra-iso-palmitate (ascorbyl tetra 2-hexyldecanoate), ascorbic acid, sodium ascorbate, dehydroascorbic acid, sodium ascorbate phosphate ester, magnesium ascorbate phosphate ester, and ascorbic acid glucoside; a group of vitamin D such as methyl hesperidin, ergocalciferol, and cholecalciferol; a group of vitamin K such as phylloquinone and farnoquinone; a group of vitamin B1 such as dibenzoyl thiamine, dibenzoyl thiamine hydrochloride, thiamine hydrochloride, thiamine cetyl hydrochloride, thiamine thiocyanate, thiamine lauryl hydrochloride, thiamine nitrate, thiamine monophosphate, thiamine lysine salt, thiamine triphosphate, thiamine monophosphate ester phosphate, thiamine monophosphate ester, thiamine diphosphate ester, thiamine diphosphate ester hydrochloride, thiamine triphosphate ester, and thiamine triphosphate ester monophosphate; a group of vitamin B6 such as pyridoxine hydrochloride, acetic acid pyridoxine, pyridoxal hydrochloride, pyridoxal 5′-phosphate, and pyridoxamine hydrochloride; a group of vitamin B12 such as cyanocobalamin, hydroxocobalamin, and deoxyadenosylcobalamin; a group of folic acids, such as folic acid and pteroyltriglutamic acid; pantothenic acids, such as pantothenic acid, calcium pantothenate, pantothenyl alcohol (panthenol), D-pantetheine, D-pantethine, coenzyme A, and pantothenyl ethyl ether; biotins, such as biotin, biocytin; and vitamin-like acting factors, such as carnitine, ferulic acid, α-lipoic acid, orotic acid, and γ-orizanol. In the case where a vitamin is mixed, the amount to be used can be appropriately selected in consideration of the feeling of use and the effect when applied to the skin, and is, for instance, about 0.001 to 30% by weight, preferably about 0.1 to 25% by weight, and more preferably about 0.5 to 20% by weight relative to the entire composition for external application to the skin of the present invention.

Examples of the above-described blood circulation-promoting ingredient include ingredients derived from plants (e.g., asian ginseng, ashitaba, arnica, ginkgo, fennel, enmeisou, holland oak, chamomile, roman chamomile, carrot (karotto), gentian, burdock, rice, hawthorn, shiitake mushroom, whitethorn, juniper, cnidium rhizome, sialid, time, clove, chenpi, Angelica acutiloba Kitagawa, Prunus persica Batsch, spruce, carrot, allium, butcher bloom, grape, tree peony, marronnier, melissa, yuzu, coix seed, rosemary, rose hip, chenpi, Angelica acutiloba Kitagawa, spruce, peach, apricot, walnut, and corn); and tocopherol nicotinate, glucosyl hesperidin, and hesperidin. In the case where a blood circulation-promoting ingredient is mixed, the amount to be used can be appropriately selected in consideration of the feeling of use and the effect when applied to the skin, and is, for instance, about 0.00001 to 10% by weight, preferably 0.0001 to 5% by weight, and more preferably about 0.001 to 5% by weight relative to the entire composition for external application to the skin of the present invention. When a plant-derived ingredient is used, the amount to be used in extract, such as essence, is about 0.00001 to 20% by weight, preferably about 0.0001 to 15% by weight, and more preferably 0.001 to 10% by weight relative to the entire composition for external application to the skin of the present invention.

Examples of the sebum-adsorbing ingredient include talc, mica, hydroxy apatite, zinc oxide, and aluminium silicate. Of these, preferred are mica, hydroxy apatite, and zinc oxide, and particularly preferred is mica. In the case where a sebum-adsorbing ingredient is mixed, the amount to be used can be appropriately selected in consideration of the feeling of use and the effect when applied to the skin, and is, for instance, about 0.001 to 35% by weight, and preferably about 0.1 to 25% by weight relative to the entire composition for external application to the skin of the present invention.

Furthermore, in addition to the above-described ingredients, ingredients commonly used in the fields of pharmaceutical products, quasi-drugs, or cosmetics may be appropriately mixed into the composition for external application to the skin of the present invention depending on the use or the dosage form. Examples of the ingredients that can be mixed into the composition include, without particular limitation, additives including a base or a carrier, a surfactant, a thickener other than the salt-resistant polymer (A), an antioxidant, a preservative, an antiseptic agent, a pH adjuster, a chelating agent, a stabilizer, a stimulus-reducing agent, a colorant, a dispersant, and a perfume. Furthermore, these ingredients may be mixed alone, or as a combination of any of two or more of them. Furthermore, the amount to be used may be appropriately determined in a range that does not impair the effect of the present invention within a conventionally known range. Moreover, an ingredient corresponding to two or more of the ingredients shown below may be added as an ingredient for imparting any of the functions.

Examples of the above-described base or carrier include a water-system base, such as water; hydrocarbon, such as liquid paraffin, squalane, vaseline, gellatinated hydrocarbon (e.g., plastibase), ozokerite, α-olefin oligomer, and light liquid paraffin; a silicone oil, such as methylpolysiloxane, cross-linking-type methylpolysiloxane, high polymerization methylpolysiloxane, cyclic silicone, alkyl-modified silicone, cross-linking type alkyl-modified silicone, amino-modified silicone, polyether-modified silicone, polyglycerin-modified silicone, cross-linking type polyether-modified silicone, cross-linking type alkylpolyether-modified silicone, silicone.alkyl chain co-modified polyether-modified silicone, silicone.alkyl chain co-modified polyglycerin-modified silicone, polyether-modified branched silicone, polyglycerin-modified branched silicone, acrylic silicon, phenyl-modified silicone, and silicone resin; higher alcohols, such as cetanol, cetostearyl alcohol, stearyl alcohol, and behenyl alcohol; cellulose derivatives, such as ethyl cellulose, hydroxy propyl cellulose, and hydroxy propyl methyl cellulose; polyvinylpyrrolidone; carrageenan; polyvinyl butyrate; polyethylene glycol; dioxane; butylene glycol adipic acid polyester; esters, such as isopropyl myristate, octyldodecyl myristate, isopropyl palmitate, cetyl palmitate, isononyl isononanoate, tetra-2-ethyl hexane acid pentaerythritol, and jojoba oil; polysaccharides other than the ingredient (B), such as dextrin and maltodextrin; lower alcohols, such as ethanol and isopropanol; and polyhydric alcohols, such as glycerin, diglycerin, propylene glycol, 1,3-butylene glycol, and isoprene glycol. Preferred is a water-system base, and more preferred is water. In the case where the composition for external application to the skin of the present invention contains water, the amount to be formulated can be appropriately selected in consideration of the feeling of use and the effect when applied to the skin, and is, for instance, about 30 to 95% by weight, preferably 50 to 95% by weight, and more preferably 60 to 95% by weight relative to the entire composition for external application to the skin of the present invention.

Examples of the above-described surfactant include various non-ion surfactants including sorbitan esters, such as sorbitan monooleate, sorbitan monoisostearate, and sorbitan monolaurate; glycerin fatty acid esters, such as POE-sorbitan monooleate, glycerin monooleate, glycerin monostearate, and glycerin monomyristate; glycerin alkyl ethers, such as monoisostearyl glyceryl ether and monomyristyl glyceryl ether; and polyglycerin fatty acid esters, such as diglycerylmonostearate, decaglyceryl decastearate, decaglyceryl decaisostearate, and diglyceryl diisostearate: or, naturally occurring surfactants, such as lecitin, hydrogenated lecitin, saponin, sodium surfactin, cholesterol, and bile acid.

Examples of the thickener other than the salt-resistant polymer (A) include vinyl-based thickeners, such as polyvinyl alcohol, polyvinylpyrrolidone, and carboxy vinylpolymer; cellulose-based thickeners, such as methyl cellulose, ethyl cellulose, hydroxy ethyl cellulose, hydroxy methyl cellulose, hydroxy propyl cellulose, hydroxy propyl methyl cellulose, carboxy methyl cellulose, and carboxy ethyl cellulose; guar gum, sclerotium gum, tamarind gum, pectin, pullulan, gelatin, locust bean gum, carrageenan, agar, acrylic acid-alkyl methacrylate copolymer, and bentonite.

Examples of the antioxidant include dibutyl hydroxy toluene, butylated hydroxyanisole, sorbic acid, sodium sulfite, ascorbic acid, ascorbic acid derivatives, tocopherol, tocopherol derivatives, erythorbic acid, and L-cysteine hydrochloride.

Examples of the preservative or antiseptic agent include benzoic acid, sodium benzoate, dehydroacetic acid, sodium dehydroacetate, isobutyl parahydroxybenzoate, isopropyl parahydroxybenzoate, butyl parahydroxybenzoate, ethyl parahydroxybenzoate, propyl parahydroxybenzoate, benzyl parahydroxybenzoate, methyl parahydroxybenzoate, phenoxy ethanol, benzyl alcohol, chlorobutanol, sorbic acid and or a salt thereof, gluconic acid chlorhexidine, methyl isothiazolinone, and iodopropynyl butylcarbamate.

Examples of the above-described pH adjuster include inorganic acids (hydrochloric acid, sulfuric acid etc.), organic acids (lactic acid, sodium lactate, citric acid, sodium citrate, succinic acid, sodium succinate etc.), inorganic bases (potassium hydroxide, sodium hydroxide etc.), and organic bases (triethanolamine, diisopropanolamine, triisopropanolamine etc.).

Examples of the chelating agent include ethylenediamine tetraacetic acid (edetic acid), ethylenediamine tetraacetate (sodium salt (sodium edetate: Japanese Pharmacopoeia, EDTA-2Na etc.), potassium salt etc.), phytic acid, gluconic acid, polyphosphoric acid, and metaphosphoric acid. Of these, sodium edetate is preferable.

Examples of the above-described stabilizer include sodium polyacrylate, dibutylhydroxytoluene, and butyl hydroxy anisole.

Examples of the stimulus-reducing agent include extract of licorice root, gum arabic, and polyvinylpyrrolidone.

<Viscosity>

The above-described composition for external application to the skin of the present invention contains a salt-resistant polymer (A), and this allows the composition to have viscosity appropriate for various uses. Specifically, there is no particular limitation as long as the composition for external application to the skin of the present invention has a viscosity (25° C.) in the range of about 5000 to 500000 mPa·s. The viscosity is preferably about 7000 to 300000 mPa·s, more preferably 10000 to 200000 mPa·s, and particularly preferably 10000 to 100000 mPa·s, because the viscosity in this range allows easy pouring of the composition from the mouth of a container that is filled with the composition at the time of use, and also allows easy spreading of the composition on the skin when applied. The composition for external application to the skin with such viscosity can be obtained mainly by appropriately selecting the type and the amount of use of the salt-resistant polymer (A) and a thickener as an additive ingredient.

Furthermore, measurement of the viscosity in the present invention is conducted using a single cylinder-type rotational viscometer (Brookfield-type viscometer) in accordance with the viscosity measurement method described in the revised Japanese Pharmacopoeia 16^{th} Edition, General Tests. In the present application, RB-80H (Toki Sangyo Co., Ltd) is used, and conditions including the rotor and the rotational speed are selected according to the instruction manual of the instrument used, and viscosity at 25° C. is measured. A description of the single cylinder-type rotational viscometer (Brookfield-type viscometer) is given below. A single cylinder-type rotational viscometer (Brookfield-type viscometer) is a viscometer that measures a torque when the cylinder in a liquid is rotated at a constant angular velocity. The device constant KB is experimentally determined in advance using viscometer standard liquids for calibrating a viscometer, so that the viscosity η of a liquid is calculated using the following equation.

η=KB×T/ω
η: Viscosity of a liquid (mPa·s)
KB: Device constant (rad/cm³)
ω: Angular velocity (rad/s)
T: Torque acting on the cylindrical surface (10⁻⁷ N·m)

<pH>

Although it is enough if the composition for external application to the skin of the present invention has a liquid property of pH 2.0 to 9.0, the composition preferably has a pH of 3.0 to 8.5, and more preferably has a pH of 5.0 to 8.0 with the object of causing less stimulus to the skin and the mucous membrane, and achieving good feeling of use when applied to the skin.

<Chemical Properties·Preparation>

The chemical properties of the composition for external application to the skin of the present invention are not particularly limited, and the composition may be in the form of a liquid, a fluid, or a semi-solid. Furthermore, the dosage forms of the preparations include liquid medicines, suspension agents, emulsions, creams, milky lotions, ointments, gels, liniments, lotions, aerosolized agents, and patch-agents of nonwoven fabric impregnated with a drug solution. Of these, milky lotions, creams, emulsions, ointments, gels, and lotions are preferable, and creams, milky lotions, ointments, and gels are particularly preferable. Furthermore, when an oil base and a water base are contained, like in emulsions, creams, and ointments, either water-in-oil type or oil-in-water type may be used, and oil-in-water type is more preferable in consideration of the effects and the feeling of use (stickiness, extension, moisture, etc.) of the composition for external application to the skin of the present invention.

Because the composition for external application to the skin of the present invention contains a salt-resistant polymer (A), the composition has appropriate viscosity and is easily applied to various sites of the skin, and because the composition contains an acidic polysaccharide (B), the composition has a superior moisturizing effect on the skin. For these reasons, the composition for external application to the skin of the present invention is applicable to various uses including moisturizing use and cosmetic use.

Furthermore, because the composition for external application to the skin contains an amphipathic compound (C), when a cosmetic is subsequently applied on the composition after the composition is applied to the skin, it is very unlikely to be occurred that a cosmetic is balled up on the skin. Hence, the composition for external application to the skin of the present invention allows the use like: after applying the composition on the skin, makeup cosmetics, such as a foundation, eyeshadow, and cheeks are subsequently applied on the portion of the skin where the composition has been applied. The methods of applying the composition for external application to the skin of the present invention and makeup cosmetics also include coating and spraying.

Furthermore, a container of any known shape may be used without limitation as a container into which the composition for external application to the skin of the present invention is filled. The material for the container is also not particularly limited, and, for instance, the composition of the present invention may be filled and provided in a container made of materials including plastics, such as polyethylene terephthalate, polyethylene naphthalate, polyarylate, polycarbonate, polyethylene, and polypropylene, as well as glass. As a container, a container made of a material such as polyethylene terephthalate, polyethylene naphthalate, or polyarylate is particularly preferable.

<Method for Producing a Composition for External Application to the Skin>

The method for producing the composition for external application to the skin of the present invention is not particularly limited, and may be produced by appropriately selecting and mixing the essential ingredients, i.e., a salt-resistant polymer (A), an acidic polysaccharide (B), and an amphipathic compound (C) and additionally the above-described other ingredients etc., and then, if necessary, emulsifying these ingredients in a usual manner.

### EXAMPLES

Hereinafter, although the present invention will be further described in detail with reference to the Examples, the present invention is not limited to these Examples.

### Preparation Examples

Compositions (test preparations) of Examples and Comparative examples were prepared in a usual manner in accordance with the formulations shown in Tables 1 to 8 below. The unit of the numerical values in the Tables is, unless otherwise specified, weight (%). Furthermore, “residual quantity” in the Tables refers to an amount relative to the total of the ingredients taken as 100% by weight.

### Test Examples

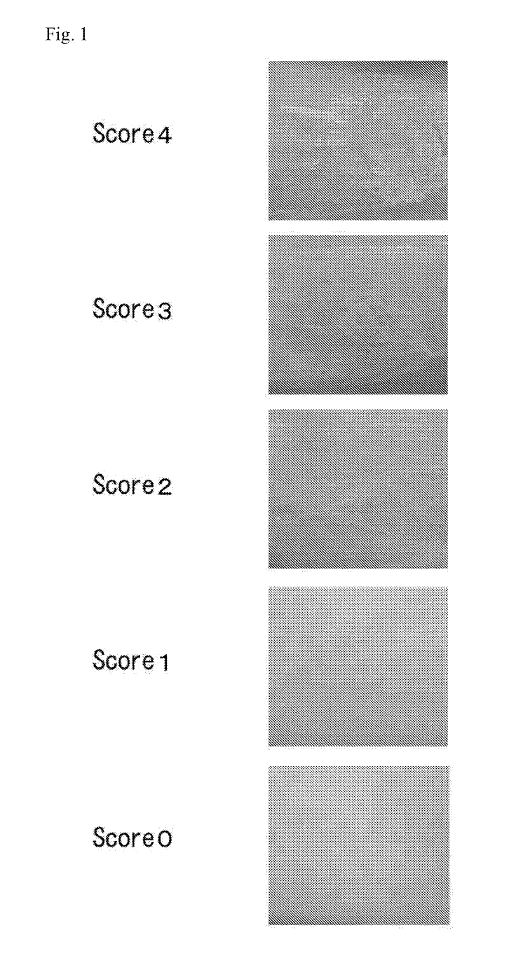

An adequate amount of each test preparation was applied (about 0.05 g) in a portion of an area of 3 cm×3 cm on the inner side of a forearm of five expert panelists, and to each portion on which the test preparation was applied, an adequate amount (about 0.02 g) of a cream foundation (Hada-labo hyaluronan BB cream 2 natural ochre: manufactured by Rohto Pharmaceutical Co., Ltd.) was further applied. The condition of the makeup on the preparation was evaluated on a score of 0 to 4 (0: no unevenness at all to 4: notable uneveness), and the numerical values were averaged. Photographs of representative conditions corresponding to the respective scores are shown in FIG. 1. It is determined that on a score of 2 or more, “unevenness” arises for which the user feels discomfort. The evaluation results are shown in Tables 1 to 8.

TABLE 1

| | Reference | Comparative | | | |
|---|---|---|---|---|---|
| | Example | Example 1 | Example 1 | Example 2 | Example 3 |
| Concentrated glycerin | 5 | 5 | 5 | 5 | 5 |
| 1,3-Butylene glycol | 10 | 10 | 10 | 10 | 10 |
| PPG-14 polyglyceryl-2 ether | — | — | 2 | — | — |
| PPG-17 buteth-17 | — | — | — | 2 | — |
| PET/PPG-150/35 copolymer | | | | | 2 |
| Sodium hyaluronate | | 0.2 | 0.2 | 0.2 | 0.2 |
| (Ammonium | 0.5 | 0.5 | 0.5 | 0.5 | 0.5 |
| acryloyldimethyltaurate/VP) | | | | | |
| copolymer | | | | | |
| pH Adjuster | adequate | adequate | adequate | adequate | adequate |
| | quantity | quantity | quantity | quantity | quantity |
| Purified water | residual | residual | residual | residual | residual |
| | quantity | quantity | quantity | quantity | quantity |
| Average score | 0 | 3.0 | 1.2 | 1.6 | 1.6 |

TABLE 2

| | Comparative | | |
|---|---|---|---|
| | Example 2 | Example 4 | Example 5 |
| Concentrated glycerin | 5 | 5 | 5 |
| 1,3-Butylene glycol | 10 | 10 | 10 |
| PPG-17 buteth-17 | — | 2 | — |
| PPG-25 sorbitol | — | — | 2 |
| Sodium hyaluronate | 0.2 | 0.2 | 0.2 |
| (Ammonium | 0.5 | 0.5 | 0.5 |
| acryloyldimethyltaurate/ | | | |
| beheneth-25 | | | |
| methaorylate) crosspolymer | | | |
| pH Adjuster | adequate | adequate | adequate |
| | quantity | quantity | quantity |
| Purified water | residual | residual | residual |
| | quantity | quantity | quantity |
| Average score | 2.4 | 1.2 | 1.4 |

TABLE 3

| | Comparative | | | | |
|---|---|---|---|---|---|
| | Example 3 | Example 6 | Example 7 | Example 8 | Example 9 |
| Concentrated glycerin | 5 | 5 | 5 | 5 | 5 |
| 1,3-Butylene glycol | 10 | 10 | 10 | 10 | 10 |
| PPG-14 polyglyceryl-2 ether | — | 2 | — | — | — |
| PPG-17 buteth-17 | — | — | 2 | — | — |
| PET/PPG-150/35 copolymer | — | — | — | 2 | — |
| PPG-25 sorbitol | — | — | — | — | 2 |
| Sodium hyaluronate | 0.2 | 0.2 | 0.2 | 0.2 | 0.2 |
| (Acrylic acid/acryloyldimethyltaurine/ | 0.5 | 0.5 | 0.5 | 0.5 | 0.5 |
| dimethylacrylamide) crosspolymer | | | | | |
| pH Adjuster | adequate | adequate | adequate | adequate | adequate |
| | quantity | quantity | quantity | quantity | quantity |
| Purified water | residual | residual | residual | residual | residual |
| | quantity | quantity | quantity | quantity | quantity |
| Average score | 2.6 | 1.4 | 1.2 | 1.2 | 1.2 |

TABLE 4

| | Comparative | |
|---|---|---|
| | Example 4 | Example 10 |
| Concentrated glycerin | 5 | 5 |
| 1,3-Butylene glycol | 10 | 10 |
| PET/PPG-150/35 copolymer | — | 0.5 |
| Sodium hyaluronate | 0.2 | 0.2 |
| (Ammonium | 0.5 | 0.5 |
| acryloyldimethyltaurate/VP) | | |
| copolymer | | |
| pH Adjuster | adequate | adequate |
| | quantity | quantity |
| Purified water | residual | residual |
| | quantity | quantity |
| Average score | 3.0 | 0.8 |

TABLE 5

| | Comparative | |
|---|---|---|
| | Example 5 | Example 11 |
| Concentrated glycerin | 5 | 5 |
| 1,3-Butylene glycol | 10 | 10 |
| PPG-17 buteth-17 | — | 3 |
| Sodium hyaluronate | 0.5 | 0.5 |
| (Ammonium | 0.5 | 0.5 |
| acryloyldimethyltaurate/ | | |
| beheneth-25 | | |
| methacrylate) crosspolymer | | |
| pH Adjuster | adequate | adequate |
| | quantity | quantity |
| Purified water | residual | residual |
| | quantity | quantity |
| Average score | 2.6 | 0.6 |

TABLE 6

| | Comparative | Comparative | |
|---|---|---|---|
| | Example 6 | Example 7 | Example 12 |
| Concentrated glycerin | 5 | 5 | 5 |
| 1,3-Butylene glycol | 10 | 10 | 10 |
| PEG/PPG/polybutylene | | 3 | |
| glycol-8/5/3 glycerine | | | |
| PPG-14 polyglyceryl-2 ether | — | — | 3 |
| Sodium hyaluronate | 0.2 | 0.2 | 0.2 |
| (Acrylic acid/ | 1 | 1 | 1 |
| acryloyldimethyltaurine/ | | | |
| dimethylacrylamide) | | | |
| crosspolymer | | | |
| pH Adjuster | adequate | adequate | adequate |
| | quantity | quantity | quantity |
| Purified water | residual | rasiduai | residual |
| | quantity | quantity | quantity |
| Average score | 2.6 | 3.6 | 1.4 |

TABLE 7

| | Compar- | | Compar- | |
|---|---|---|---|---|
| | ative | Example | ative | Example |
| | Example 8 | 13 | Example 9 | 14 |
| Concentrated glycerin | 5 | 5 | 5 | 5 |
| 1,3-Butylene glycol | 10 | 10 | 10 | 10 |
| PPG-17 buteth-17 | 2 | 2 | | |
| Sodium hyaluronate | 0.05 | 0.05 | 0.1 | 0.1 |
| (Ammonium | 1 | 1 | 1 | 1 |
| acryloyldimethyltaurate/ | | | | |
| beheneth-25 | | | | |
| methacrylate) | | | | |
| crosspolymer | | | | |
| pH Adjuster | adequate | adequate | adequate | adequate |
| | quantity | quantity | quantity | quantity |
| Purified water | residual | residial | residual | residial |
| | quantity | quantity | quantity | quantity |
| Average score | 2.2 | 0.6 | 2.6 | 0.6 |

TABLE 8

| | Comparative | |
|---|---|---|
| | Example 10 | Example 15 |
| Concentrated glycerin | 0 | 5 |
| 1,3-Butylene glycol | 10 | 10 |
| PEG/PPG/polybuthylene | 1 | — |
| glycol-8/5/3 glycerine | | |
| PPG-25 sorbitol | — | 1 |
| Xanthan gum | 0.5 | 0.5 |
| (Ammonium | 0.5 | 0.5 |
| acryloyldimethyltaurate/ | | |
| VP) copolymer | | |
| pH Adjuster | adequate | adequate |
| | quantity | quantity |
| Purified water | residual | residual |
| | quantity | quantity |
| Average score | 2.4 | 1.4 |

According to Table 1, no balling up of a cosmetic on the skin occurred at all in the preparation (Reference Example) where only (Ammonium acryloyldimethyltaurate/VP) copolymer was mixed, without sodium hyaluronate, to concentrated glycerin and 1,3-butylene glycol, which constitute a base, whereas balling up of a cosmetic on the skin occurred in the preparation (Comparative Example 1) where sodium hyaluronate was further added. However, significant improvement of balling up of a cosmetic on the skin was observed in the preparations (Examples 1 to 3) where an amphipathic compound (C), such as PPG-14 polyglyceryl-2 ether, PPG-17 buteth-17, or PEG/PPG-150/35 copolymer, was further added.

According to Table 2, balling up of a cosmetic on the skin occurred in the preparation (Comparative Example 2) where no amphipathic compound (C) was mixed, whereas significant improvement of balling up of a cosmetic on the skin was observed in the preparations (Examples 4 and 5) where PPG-17 buteth-17 or PPG-25 sorbitol was mixed.

The same tendency was observed in Tables 3 to 7, and it was also found that various salt-resistant polymers (A) can be used in the present invention. Furthermore, according to Table 6, balling up of a cosmetic on the skin occurred in the preparation (Comparative Example 7) where PEG/PPG/polybutylene glycol-8/5/3 glycerin, which is an amphipathic compound but does not correspond to the ingredient (C), was mixed.

According to Table 8, mixing a salt-resistant polymer (A) and an acidic polysaccharide (B) in the form of xanthane gum resulted in balling up of a cosmetic on the skin, and even further mixing of an amphipathic compound that does not correspond to an amphipathic compound (C) of the present invention failed to prevent balling up of a cosmetic on the skin (Comparative Example 10), whereas mixing an amphipathic compound (C) of the present invention resulted in significant improvement of balling up of a cosmetic on the skin (Example 15).

### Formulation Examples

Formulation examples of the composition for external application to the skin of the present invention are shown below. The numerical values given below are in the unit of weight (%).

TABLE 9
Formulation Example 1 Lotion

| | |
|---|---|
| Concentrated glycerin | 3.0 |
| Diglycerin | 2.0 |
| 1,3-Butylene glycol | 5.0 |
| (Ammonium | 0.2 |
| acryloyldimethyltaurate/ | |
| vinylpyrrolidone) copolymer | |
| Sodium hyaluronate | 0.05 |
| PPG-25 sorbitol | 0.5 |
| Polyoxyethylene | 0.8 |
| hydrogenated castor oil | |
| Glucosylceramide | 0.005 |
| Antiseptic agent (methyl | adequate |
| parahydroxybenzoate, | quanitity |
| methylisothiazolinone) | |
| pH Adjuster | adequate |
| | quanitity |
| Perfume | adequate |
| | quanitity |
| Purified water | residual |
| | portion |
| Total | 100. |

TABLE 10
Formulation Example 2 Gel

| | |
|---|---|
| Hydroxyethyl urea | 8.0 |
| Dipropylene glycol | 5.0 |
| (Hydroxyethyl acrylate/Na | 1.8 |
| acryloyldimethyltaurate) copolymer | |
| Sodium hyaluronate | 0.2 |
| Gellan gum | 0.1 |
| Polyoxypropylene methyl glucoside | 0.2 |
| PPG-17 buteth-17 | 1.5 |
| Sorbitan monostearate | 1.0 |
| Triethyl citrate | 1.0 |
| Antiseptic agent (methyl parahydroxybenzoate, | adequate |
| methylisothiazolinone, | quantity |
| phenoxy ethanol) | |
| pH Adjuster | adequate |
| | quantity |
| Perfume | adequate |
| | quantity |
| Purified water | residual |
| | portion |
| Total | 100. |

TABLE 11
Chemical formulation 3 Emulsion

| | |
|---|---|
| Concentrated glycerin | 5.0 |
| Dipropylene glycol | 2.0 |
| 1,2-Pentanediol | 3.0 |
| (Ammonium acryloyldimethyltaurate/beheneth-25 | 1.2 |
| methacrylate) cross-copolymer | |
| Xanthane gum | 0.5 |
| Sodium hyaluronate | 0.02 |
| Oligo hyaluronic acid | 0.1 |
| PPG-14 polyglyceryl-2 ether | 1.5 |
| Polyoxyethylene cetyl ether | 0.5 |
| Polyoxyethylene hydrogenated castor oil | 0.3 |
| Glyceryl monostearate | 1.0 |
| Tri(capryl•caprylic acid)glyceryl | 5.0 |
| Methylpolysiloxane | 1.0 |
| Chelating agent | adequate |
| | quantity |
| Antiseptic agent (methyl parahydroxybenzoate, | adequate |
| propyl parahydroxybenzoate, | quantity |
| phenoxy ethanol) | |
| pH Adjuster | adequate |
| | quantity |
| Perfume | adequate |
| | quantity |
| Purified water | residual |
| | portion |
| Total | 100. |

TABLE 12
Formulation Example 4 Emulsion

| | |
|---|---|
| Concentrated glycerin | 5.0 |
| 1,3-Butylene glycol | 5.0 |
| (Ammonium | 0.3 |
| acryloyldimethyltaurate/vinylpyrrolidone) | |
| copolymer | |
| (Ammonium acryloyldimethyltaurate/beheneth-25 | 0.5 |
| methacrylate) cross-copolymer | |
| Sodium hyaluronate | 0.01 |
| Acetylated sodium hyaluronate | 0.01 |
| Xanthan gum | 0.1 |
| Carrageenan | 0.1 |
| PPG-16 glyceryl ether | 0.5 |
| PPG-17 buteth-17 | 0.5 |
| Polyoxyethylene behenyl ether | 1.4 |
| Sorbitan monoisostearate | 1.6 |
| Squalene | 6.0 |
| Glyceryl tri-2-ethylhexanoate | 3.0 |
| Meadowfoam oil | 0.5 |
| Methyl polysiloxane | 0.3 |
| Cetanol | 0.2 |
| Hydrolyzed collagen powder | 0.1 |
| Chelating agent | adequate |
| | quantity |
| Antiseptic agent (methyl parahydroxybenzoate, | adequate |
| propyl parahydroxybenzoate, phenoxy ethanol) | quantity |
| pH Adjuster | adequate |
| | quantity |
| Perfume | adequate |
| | quantity |
| Purified water | residual |
| | portion |
| Total | 100. |

TABLE 13
Formulation Example 5 Gel cream

| | |
|---|---|
| Diglycerin | 4.0 |
| 1,3-Butylene-glycol | 10.0 |
| 1,2-Hexanediol | 2.0 |
| (Ammonium | 0.5 |
| acryloyldimethyltaurate/vinylpyrrolidone) | |
| copolymer | |
| Sodium hyaluronate | 0.5 |
| Sclerotium gum | 0.2 |
| Polyoxypropylene methyl glucoside | 0.5 |
| PEG/PPG-150/35 copolymer | 2.5 |
| Squalene | 2.0 |
| Glyceryl tri-2-ethylhexanoate | 5.0 |
| Decamethylcyclopentasiloxane | 3.0 |
| Methylpolysiloxane | 1.0 |
| Behenyl alcohol | 1.0 |
| Stearyl alcohol | 1.0 |
| Cetearyl glucoside/Cetearyl alcohol | 1.0 |
| Hydrolyzed collagen powder | 0.05 |
| Sodium metabisulfite | 0.02 |
| Chelating agent | adequate |
| | quantity |
| Antiseptic agent (methyl parahydroxybenzoate, | adequate |
| propyl parahydroxybenzoate, phenoxy ethanol) | quantity |
| pH Adjuster | adequate |
| | quantity |
| Perfume | adequate |
| | quantity |
| Purified water | residual |
| | portion |
| Total | 100. |

TABLE 14
Formulation Example 6 Cream

| | |
|---|---|
| Hydroxyethyl urea | 3.0 |
| Concentrated glycerin | 5.0 |
| 1,3-Butylene-glycol | 10.0 |
| (Hydroxyethyl acrylate/Na | 1.2 |
| acryloyldimethyltaurate) copolymer | |
| Sodium hyaluronate | 0.3 |
| Gellan gum | 0.2 |
| Polyacrylic acid amide | 0.1 |
| Sorbitol | 1.0 |
| PEG/PPG-150/35 copolymer | 3.0 |
| N-stearoyl sodium glutamate | 0.2 |
| Glyceryl monostearate | 0.5 |
| Sucrose fatty acid ester | 0.3 |
| Sorbitan monostearate | 0.2 |
| Liquid paraffin | 2.0 |
| α-Orefin oligomer | 4.0 |
| tri-2-Ethyl hexanoate pentaerythritol | 5.0 |
| White petrolatum | 3.0 |
| Shea butter | 1.5 |
| Methylpolysiloxane | 2.0 |
| Chelating agent | adequate |
| | quantity |
| Antiseptic agent (methyl parahydroxybenzoate, propyl | adequate |
| parahydroxybenzoate, butyl carbamate iodide propynyl) | quantity |
| PH Adjuster | adequate |
| | quantity |
| Perfume | adequate |
| | quantity |
| Purified water | residual |
| | portion |
| Total | 100. |

1. A composition for external application to skin comprising:
(A) a salt-resistant polymer;
(B) an acidic polysaccharide; and
(C) an amphipathic compound represented by general formula (1) below:
Z—[O-{(EO)ₐ(PO)_{b}}(AO)_{c}—H]ₙ (1)
wherein:
n is an integer of 1 to 10;
Z is a hydrogen atom, or a group obtained by removing n number of hydroxy group(s) from a hydroxy compound of a carbon number of 1 to 30;
EO is an ethylene oxide group;
PO is a propylene oxide group;
AO is an alkylene oxide group of a carbon number of 2 to 5;
a, b, and c each denote an average addition molar number of respective alkylene oxide groups, and are independently 0 to 200; provided that not all of a, b, and c are 0 at the same time; when n is 2 or more, respective pluralities of a's, b's, c's, and AOs may be the same as or different from one another; and when n is 3, the carbon number of AO is 2, 3, or 5; and when Z is a hydrogen atom, n is 1; and
wherein the respective alkylene oxide groups in { } may be added randomly or in blocks.

2. The composition for external application to skin according to claim 1, wherein the amphipathic compound is a compound represented by general formula (2) below:
Z—[O-{(EO)ₐ(PO)_{b}}—H]ₙ (2)
wherein:
n is an integer of 1 to 10;
Z is a hydrogen atom, or a group obtained by removing n number of hydroxy group(s) from a hydroxy compound of a carbon number of 1 to 30;
EO is an ethylene oxide group;
PO is a propylene oxide group;
a and b each denote an average addition molar number of respective alkylene oxide groups, and are independently 0 to 200; provided that not all of a and b are 0 at the same time, and when n is 2 or more, respective pluralities of a's and b's may be the same as or different from one another; and when Z is a hydrogen atom, n is 1; and
wherein the respective alkylene oxide groups in { } may be added randomly or in blocks.

3. The composition for external application to skin according to claim 1 or 2, wherein Z in the above-described general formula (1) or (2) is a hydrogen atom, or, a group obtained by removing n number of hydroxy group(s) from an alkyl monoalcohol of a carbon number of 4 to 24, glycerin, trimethylolpropane, erythritol, pentaerythritol, alkylglycoside, diglycerin, xylitol, dipentaerythritol, sorbitol, inositol, sucrose, trehalose, or maltitol.

4. The composition for external application to skin according to claim 1, wherein Z in the above-described general formula (1) or (2) is a hydrogen atom, or a group obtained by removing a hydroxy group from an alkyl monoalcohol of a carbon number of 4 to 24, a group obtained by removing 1 to 6 hydroxy groups from sorbitol, or a group obtained by removing 1 to 4 hydroxy groups from diglycerin.

5. The composition for external application to skin according to claim 1, wherein the acidic polysaccharide (B) is an acidic mucopolysaccharide, xanthane gum, or gellan gum.

6. The composition for external application to skin according to claim 1, wherein the salt-resistant polymer (A) is a polymer having, on a side chain thereof, dimethyl taurine or a salt thereof.

7. The composition for external application to skin according to claim 6, wherein the salt-resistant polymer (A) is at least one selected from the group consisting of (hydroxyethyl acrylate/Na acryloyldimethyltaurate) copolymer, (ammonium acryloyldimethyltaurate/vinylpyrrolidone) copolymer, (ammonium acryloyldimethyltaurate/beheneth-25 methacrylate) cross-copolymer, (ammonium acryloyldimethyltaurate/steareth-25 methacrylate) crosspolymer, and (acrylic acid/acryloyldimethyltaurine/dimethylacrylamide) crosspolymer.

8. A method for applying makeup, comprising applying to skin the composition for external application to skin according to claim 1, and subsequently applying a makeup cosmetic on the skin.
